(19) United States
(12) Patent Application Publication
(10) Pub. No.: US 2016/0089411 A1
(43) Pub. Date: Mar. 31, 2016

(54) METHOD AND COMPOSITION FOR TREATING SYMPTOMS OF SICKLE CELL DISEASE

(71) Applicant: Umm Al-Qura University, Makkah (SA)

(72) Inventor: Saeed Saeed Alghamdi, Makkah (SA)

(73) Assignee: Umm Al-Qura University, Makkah (SA)

(21) Appl. No.: 14/414,226

(22) PCT Filed: Sep. 30, 2014
(86) PCT No.: PCT/IB2014/001969

§ 371 Date: Jan. 12, 2015

(51) Int. Cl. A61K 36/725; A61K 36/63; A23L 2/52; A61K 36/31

(52) U.S. Cl. CPC A61K 36/725; A61K 36/31; A61K 36/63; A23L 2/52; A23V 2002/00

(57) ABSTRACT

Disclosed herein is a nutraceutical composition comprising dried Ziziphus mauritiana leaves, dried Lepidium sativum leaves, dried olive leaves at specific weight ratios and a specific amount of olive oil. The disclosed composition has been shown to effectively treat with minimal side effects, the common symptoms of sickle cell disease (SCD) including severe pain, bacterial infection, inflammation, wounds, fever, cough, shortness of breath. Various embodiments of methods of treating SCD patients using the same composition and methods of preparing the composition are also provided.

### BACKGROUND OF THE INVENTION

1. Technical Field

The present invention relates to a composition and a method of treating various symptoms of sickle cell disease. Specifically, the present invention relates to a nutraceutical composition and a method of using thereof for the treatment and management of the symptoms of the disease.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Sickle cell disease (SCD) is a group of hereditary blood disorders. It affects millions of people throughout the world and is particularly common among those whose ancestors came from sub-Saharan Africa; Spanish-speaking regions in the Western Hemisphere (South America, the Caribbean, and Central America); Saudi Arabia; Indian; and Mediterranean countries such as Turkey, Greece and Italy. It is estimated that sickle cell disease affects 90,000 to 100,000 Americans. Life expectancy is shortened, although with careful management of the disease, patients can live into their 70 s and beyond.

SCD is characterized by red blood cells that assume an abnormal, rigid, sickle shape. Healthy red blood cells are round, and they move through small blood vessels to carry oxygen to all parts of the body. For a sickle cell disease patient, his or her red blood cells die early, causing a constant shortage thereof. Also, when these sickle cells travel through small blood vessels, they get stuck and clog the blood flow. This can cause excruciating pain among other complications that are discussed below.

Different forms of SCD include the homozygous sickle-cell anemia (HbSS), the heterozygous sickle-cell trait (HbAC), and the compound heterozygous sickle-hemoglobin C (HbSC), sickle-hemoglobin D (HbSD), sickle-hemoglobin E (HbSE), sickle-hemoglobin O (HbSO), sickle-beta-plus-thalassemia (HbS/β⁺), sickle-beta-zero-thalassemia)(HbS/β⁰). Among these various forms of sickle cell disease, sickle cell anemia is usually the most severe.

People with SCD show first signs of the disease during their first year of life, usually around 5 months of age. Symptoms and complications can vary for each person and can range from mild and severe. Many of such symptoms and complications can also be chronic. Pain, as mentioned earlier, is the most common symptom of SCD, and the top reason that people with SCD are admitted to the emergency room or hospital. When sickle cells get stuck in blood vessels and clog the blood flow, pain can start suddenly, and last for any length of time. The terms “sickle cell crisis” or “sickling crisis” are used to describe these acute pain spells. Other major symptoms of SCD include, for example, fatigue, dizziness, loss of vision, bacterial infections, inflammation, stroke and difficulty in breathing. These symptoms are associated with the secondary complications that SCD patients encounter, for example, anemia, head-foot syndrome, acute chest syndrome and leg ulcers.

The only known cure to SCD is bone marrow or stem cell transplants. Unfortunately, these procedures are very risky and can have serious side effects, including death. Therefore, it is important for SCD patients to manage the disease and to treat its symptoms accordingly. Examples of these management and treatment methods are blood transfusion (for severe anemia, infections, acute chest syndrome), hydroxyurea, antibiotics (for wounds and infections), analgesics (including over-the-counter non-steroidal anti-inflammatory drugs like ibuprofen and aspirin as well as opioids like morphine), creams and ointments (for leg ulcers). These methods are often accompanied by adverse side effects, and can also sometimes be ineffective.

Accordingly, there exists a considerable need for safe agents that can effectively manage the many symptoms of SCD. Nutraceuticals, in recent decades, have emerged as an increasingly popular option for the treatment and prevention of various diseases. Nutraceuticals, a term coined as a portmanteau of the words “nutrition” and “pharmaceutical”, refer to products range isolated nutrients, dietary supplements and herbal products that provide medical and health benefits including prevention and treatment of disease (Kalra, E K, 2003, “Nutraceutical—definition and introduction,” AAPS pharmSci 5(3): 27-28; Ramaa, C S, Shirode A R, Mundada A S, and Kadam V J, 2006, “Nutraceuticals—an emerging era in the treatment and prevention of cardiovascular diseases,” Curr Pharm Biotechnol 7(1): 15-23—each incorporated herein by reference in its entirety). It is based upon a worldwide acceptance of recognition link between “nutrition” and “health” that the concept of nutraceuticals was evolved. Some examples of well-regarded nutraceuticals include garlic, omega-3-fatty acids, soy products, plant sterols, flavonoids and probiotics.

The Indian jujube or its scientific name, Ziziphus mauritiana, is a tropic fruit tree belonging to the family Rhamnaceae. The species is believed to have originated in the Indo-Malaysian region of Southeast Asia but has since widely naturalized from Southern Africa through the Middle East to the Indian subcontinent, China, and into Australasia and Pacific Islands. The fruit, rich in vitamin C, can be eaten raw, stew, pickled or used in beverages. The fruit is also sometimes applied on cuts, ulcers, and in pulmonary ailments and fevers. The leaves are traditionally consumed by human as vegetables in Indonesia or as animal feed in other parts of the world where the plant is found in abundance. Recent biochemical characterization of the Z. mauritiana leaf revealed presence of different types of secondary metabolites such as glycosides, tannins, phenols and saponins (Najafi, S, 2013, “Phytochemical screening and antibacterial activity of leaf extract of Ziziphus mauritiana Lam.,” International Research Journal of Applied and Basic Sciences 4(11): 3274-76—incorporated herein by reference in its entirety). Saponins have been promoted commercially as nutraceuticals and ginseng saponin has been shown to have therapeutic effects on skin wound healing (Kim Y S, Cho I H, Jeong M J, Jeong S J, Nah S Y, Cho Y S, Kim S H, Go A, Kim S E, Kang S S, Moon C J, Kim J C, Kim S H, and Bae C S, 2011, “Therapeutic effect of total ginseng saponin on skin wound healing,” J Ginseng Res 35(3): 360-67—incorporated herein by reference in its entirety).

The garden cress Lepidium sativum has been considered an important medicinal plant since the Vedic area in India. The whole plant, leaves, root and seed can be used for a multitude of medicinal purposes. The seed and its oil, in particular, have been found useful in the treatment of asthma, coughs, bronchitis and the improvement of lung function (Rehman, N U, Khan, A U, Alkharfy, K M, and Gilani, A H, 2012, “Pharmacological basis for the medicinal use of Lepidium sativum in airways disorders,” Evidence-Based Complementary and Alternative Medicine—incorporated herein by reference in its entirety).

The oil derived from Olea europaea, or simply olive oil, is commonly used in cooking, religions, cosmetics, pharmaceuticals, soaps, lubricants, and as a fuel for traditional lamps throughout the world. Consumption of olive oil has been linked to the lowering of heart disease risk factors such as blood cholesterol levels and LCL cholesterol oxidation. Studies also suggest that olive may also influence inflammatory, thrombotic, hypertensive and vasodilatory mechanisms. Olive oil contains phenolics such as esters of tyrol and hydroxytyrosol, including oleocanthal and oleuropein. Olive oil is a source of at least 30 phenolic compounds that are postulated to having anticancer, antioxidant and anti-aging effects.

While olive oil is renowned for its flavor and health benefits, the leaf of the same crop has been used medicinally for various purposes, Olive leaf and olive leaf extracts are marketed as anti-aging, immunostimulator and antibiotic agents. The primary active compounds in unprocessed olive leaf are believed to be, like olive oil, the antioxidants oleuropein and hydroxytyrosol, as well as other polyphenols and flavonoids, including oleocanthal.

It remains a mission for those skilled in the nutritional art to establish a composition and a method of using the same by which symptoms of SCD can be managed, treated or relieved with minimal side effects. Disclosed embodiments of the present invention overcome the shortcomings of the prior art as described herein.

### BRIEF SUMMARY OF THE INVENTION

The foregoing paragraphs have been provided by way of general introduction, and are not intended to limit the scope of the following claims. The described embodiments, together with further advantages, will be best understood by reference to the following detailed description taken in conjunction with the accompanying drawings.

According to a first aspect, the present invention is directed to a nutraceutical composition comprising dried Ziziphus mauritiana leaves, dried Lepidium sativum leaves, dried olive leaves at specific weight ratios and a specific amount of olive oil. The disclosed composition has been shown to effectively treat with minimal side effects, the common symptoms of sickle cell disease (SCD) including severe pain, bacterial infection, inflammation, wounds, fever, cough, shortness of breath.

According to a second aspect, the present invention relates to a method of treating SCD patients using the nutraceutical composition.

According to a third aspect, the present invention relates to a nutraceutical beverage based upon the disclosed composition.

Various embodiments of methods of treating SCD symptoms and methods of preparing the composition are also described in the present invention.

### DETAILED DESCRIPTION OF THE EMBODIMENTS

For the treatment of common major symptoms associated with sickle cell disease (SCD), a nutraceutical composition comprising specific amounts of dried leaves of Ziziphus mauritiana, seeds of Lepidium sativum, dried olive leaves and olive oil, may be effectively used. These major symptoms include, but are not limited to, severe pain, bacterial infection, inflammation, wounds, fever, cough, shortness of breath. In other words, the composition disclosed herein is designed to possess analgesic, anti-inflammatory, anti-bacterial, wound-healing properties and to also improve lung and breathing functions.

In one embodiment, the aforementioned components for the preparation of the disclosed composition may be commercially available in pharmaceutically acceptable purity. Olive oil, due to its global popularity as for culinary uses, can be readily supplied commercially. In the United States, olive leaf extracts in powder or capsule forms are sold at pharmacies such as Walgreens, and retailers of nutritional supplements such as The Vitamin Shoppe, or even online on Amazon. Seeds of Lepidium sativum and leave extracts of Ziziphus mauritiana may be found online on Amazon and eBay or on site at ethnic grocery stores.

In one embodiment, in areas with a tropical climate where Ziziphus mauritiana shrubs or trees can be easily cultivated, fresh leaves may be obtained. In United States, for example, Z. mauritiana trees are found in southern Florida. In this case, leaves that are freshly cut from their trees may be washed gently in cold running water to remove any soil, dust, bugs or other foreign material. The washed leaves are drained thoroughly on absorbent towels or they can be hung upside in the sun until the water evaporates. The leaves are stripped off their stalks once they have drained and dried. The dried leaves may be stored in rigid, airtight and light-proof containers.

In another embodiment, the freshly cut leaves of Ziziphus mauritiana may be air-dried. The leaves may be spread out on window screens and turned occasionally to ensure even drying.

In another embodiment, small amounts of Ziziphus mauritiana leaves may be dried in a microwave. A single layer of cleaned leaves are laid upon dry paper towels with sufficient space between them and placed in the microwave for 1 to 2 minutes on high power. Drying will vary with the moisture content of the leaves and wattage of the microwave. If the leaves are not brittle when touched, they may be reheated for 30 seconds and tested for brittleness again. This reheating process may be repeated as many times as needed to ensure that the leaves are completely dry.

In another embodiment, conventional ovens may be used to dry the Ziziphus mauritiana leaves. The leaves are placed on a cookie sheet or shallow pan no more than an inch deep in an open oven at low heat (less than 180 degrees Fahrenheit or 82.2 degrees Celsius) for 2 to 4 hours.

In yet another embodiment, silica gel or non-iodized table salt can be used to dry or Ziziphus mauritiana leaves. Cleaned leaves are blotted dry before they are placed in a tray or shallow pan of the silica gel or salt. After the leaves have dried in approximately 2 to 4 weeks, the leaves are removed from the drying material. Excess material is shaken off and the leaves are stored in glass containers. Before using, these leaves should be rinsed thoroughly in clear, cold water.

In yet another embodiment the Ziziphus mauritiana leaves may be removed from the plants, washed and spread thinly on screens to dry, avoiding exposure to sunlight or other alternative bright light sources.

Whatever method is chosen to dry the leaves, it is important to dry the leaves as thoroughly as possible in order to reduce the moisture content to a minimum. Water content is preferably no more than 5% by weight based on the weight of the leaves. Care must also be taken to prevent loss of flavor, oils and color in order to preserve the quality of the leaves. In the present invention, thorough drying is also pivotal to ensure the precision in the preparation of the dosage of the composition described herein. To retain some green leaf coloring, if desired, the Ziziphus mauritiana leaves may be dried in the dark in paper bags or away from sunlight.

All of the aforementioned drying methods may be used upon Lepidium sativum seeds and olive leaves, when these herbs are acquired fresh.

To prepare the disclosed novel composition, equal amounts of dried Ziziphus mauritiana leaves, dried Lepidium sativum seeds and dried olive leaves are weighed. In other words, the weight ratio of dried Ziziphus mauritiana leaves to dried Lepidium sativum seeds and dried olive leaves is preferably fixed at 1:1:1. In one exemplary embodiment, 6.6 g of dried Ziziphus mauritiana leaves, 6.6 g of dried Lepidium sativum seeds and 6.6 g of dried olive leaves, are used, so that the total weight of the dried herbs is about 20 g. In other embodiments the amounts of the ingredients may be within ranges such that the amount of any individual component is present in an amount of from 0.1 to 10 times by weight of any of the other two aforementioned components, preferably 0.2-8, 0.4-6, 0.6-4, 0.8-2 times by weight of any of the other two aforementioned components.

Next, each of the weighed components of the disclosed composition is ground individually. This may be achieved in several ways, for example, with an electric food grinder or processor, blender as well as mechanically with a set of mortar and pestle. The goal of this step is to attain the Ziziphus mauritiana leaves, Lepidium sativum seeds and olive leaves in fine powder form that promotes easy absorption by the body.

The freshly ground Ziziphus mauritiana leaves, Lepidium sativum seeds and olive leaves are then mixed together thoroughly. These components may be mixed with an electrical device, or manually with a spatula.

300 ml or 10.1 fl oz. of olive oil is measured and added to the herbal mixture described above and mixed thoroughly again to produce the composition claimed in this present invention. The composition can be stored a clean container at a cool place. In other embodiments the olive oil is used with the herbal mixture described above in an amount of from 50 to 500 ml, preferably 100 to 400 or 200 to 300 ml.

Thus, to prepare the disclosed composition that is made up of 20 g of total weight of the dried herbs, one recipe is as follows:

6.6 g of ground dried Ziziphus mauritiana leaves

6.6 g of ground dried Lepidium sativum seeds

6.6 g of ground dried olive leaves (Olea europaea)

300 ml of olive oil

The composition may be prepared on a larger or a smaller scale, depending on individual cases and needs, preferably as long as the weight ratio of the three dried herbs is maintained at 1:1:1 and the amount of olive oil is adjusted accordingly. For example, in one embodiment, the composition may be prepared using 33.66 g of dried Ziziphus mauritiana leaves, 33.66 g of dried Lepidium sativum seeds, 33.66 g of dried olive leaves and 1500 ml (50.7 fl oz.) of olive oil. In another embodiment, 100 mg of each of dried Ziziphus mauritiana leaves, dried Lepidium sativum seeds and dried olive leaves is used, and 4.5 ml (0.15 fl oz.) of olive oil is added to the herbs.

In another embodiment, the ratio by weight of dried Ziziphus mauritiana leaves to dried Lepidium sativum seeds and dried olive leaves may be x:y:z, where x=1, 2, 3, 4, 5; y=1, 2, 3, 4, 5 and z=1, 2, 3, 4, 5.

In an alternative embodiment, any of the dried Ziziphus mauritiana leaves, dried Lepidium sativum seeds and dried olive leaves used to prepare the disclosed composition may already be in powder form, for example, olive leaf extract and ground Lepidium sativum seeds. In this case, the grinding step of preparation process will not be necessary.

The olive oil used in the preparation of the described nutraceutical composition may be of any retail grade that is fit for human consumption with free fatty acid content of not more than 2 g per 100 g (2%). The olive oil, besides its own health benefits, serves as an organic solvent for the numerous compounds found in the other dry constituents. For example, the process of saponin extraction is enhanced by means of the use of an organic solvent. Moreover, the olive oil creates a sterile environment for the composition. It also acts as a lubricant, easing a patient's oral consumption of the composition by making it easy to swallow.

The present invention is also directed to a method of treating common major symptoms of SCD by oral administration of the disclosed nutraceutical composition. In oral administration, a daily dosage of 0.07 g per kg to 0.7 g per kg of body weight is effective for treating severe pain, bacterial infection, inflammation, wounds, fever, cough, shortness of breath, hemoglobin concentration, frequency of hospitalization, and frequency of pain crises. A human subject diagnosed with any form of SCD or showing symptoms of SCD is given a therapeutically effective amount of the nutraceutical composition twice a day—once early in the morning before breakfast and 12 hours after that). A therapeutically effective amount of the nutraceutical composition refers to 0.07 g per kg to 0.7 g per kg (of body weight. This dosage was tested upon 30 volunteers who are SCD patients. The subjects were administered two dosages of the nutraceutical composition at 0.7 g per kg of body weight, every day, for two months. At the end of the two-month course, all subjects reported a sharp decrease in chronic pain, infection, swelling, inflammation and fever. The white blood cell (WBC) count and hemoglobin levels for the 30 SCD patients (before and after treatment with disclosed nutraceutical composition for 2 months at the dosage 0.7 g per kg of body weight of the patient), positive controls (30 untreated SCD patients) and negative controls (30 healthy individuals) were monitored and the mean average values are presented in Table 1. Those suffering from leg ulcers reported a marked improvement in wound-healing and acute chest syndrome patients saw an overall improvement of breathing and lung functions. All treat patients noted a dramatic decrease in severe pain resulted from hemolysis (rupture of red blood cells).

TABLE 1

| | WBC count | WBC count | | |
|---|---|---|---|---|
| | before treatment | after treatment | Control | Control |
| Patient | (×10⁻⁹/L) | (×10⁻⁹/L) | positive | negative |
| 1 | 22.0 | 10.0 | 32.0 | 5.5 |
| 2 | 19.0 | 7.3 | 35.0 | 6.1 |
| 3 | 23.0 | 8.0 | 29.1 | 7.1 |
| 4 | 30.0 | 9.0 | 36.0 | 9.0 |
| 5 | 22.0 | 6.1 | 27.0 | 8.2 |
| 6 | 21.0 | 8.0 | 28.1 | 6.0 |
| 7 | 29.0 | 9.0 | 22.0 | 8.0 |
| 8 | 31.0 | 7.0 | 18.0 | 9.3 |
| 9 | 30.0 | 6.7 | 24.3 | 5.0 |
| 10 | 26.0 | 5.0 | 31.0 | 6.3 |
| 11 | 33.0 | 10.0 | 31.0 | 7.0 |
| 12 | 29.0 | 6.6 | 38.0 | 7.1 |
| 13 | 27.0 | 5.6 | 29.2 | 6.4 |
| 14 | 19.0 | 5.0 | 38.0 | 8.0 |
| 15 | 22.0 | 9.3 | 24.0 | 9.4 |
| 16 | 25.0 | 6.0 | 36.0 | 7.1 |
| 17 | 23.0 | 8.0 | 30.0 | 8.4 |
| 18 | 34.0 | 7.5 | 31.0 | 9.1 |
| 19 | 27.0 | 6.0 | 23.0 | 10.0 |
| 20 | 19.0 | 6.1 | 34.0 | 9.3 |
| 21 | 24.0 | 8.1 | 27.0 | 10.0 |
| 22 | 26.0 | 6.0 | 31.7 | 8.0 |
| 23 | 21.0 | 6.0 | 29.0 | 6.8 |
| 24 | 28.0 | 5.0 | 33.0 | 7.0 |
| 25 | 27.0 | 5.9 | 23.0 | 5.9 |
| 26 | 32.0 | 9.1 | 31.3 | 7.5 |
| 27 | 32.0 | 4.9 | 31.4 | 9.0 |
| 28 | 28.0 | 7.0 | 36.0 | 6.9 |
| 29 | 18.0 | 4.8 | 34.0 | 10.0 |
| 30 | 28.0 | 7.0 | 30.3 | 6.1 |

Chronic symptoms of SCD, if ever, take a long time to treat and alleviate. For example, it takes about 3 months to graft SCD leg ulcers. The procedure is complicated, may take multiple attempts and failure rates are high. Conventional methods treat leg ulcers topically and surgically. The invention described herein presents an unprecedented and successful method to treat these lesions with an oral medication. However, with that being said, in one embodiment the nutraceutical composition may be applied topically onto leg ulcers.

Yet in another embodiment, the nutraceutical composition disclosed at the prescribed dosage may be diluted into a beverage and flavored with sweeteners.

In another aspect, the invention is directed to an oral solid dosage form that can be a tablet, a caplet, a gelcap, or a capsule that includes the nutraceutical composition and, optionally, one or more pharmaceutically acceptable excipients as is known in the art. Pharmaceutically acceptable excipients assist or make possible the formation of a dosage form for a bioactive material and include diluents, binding agents, lubricants, glidants, disintergrants, coloring agents, and flavorants and nutrients. An excipient is pharmaceutically acceptable if, in addition to performing its desired function, it is non-toxic, well tolerated upon ingestion, and does not interfere with absorption of bioactive materials.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public. further advantages, will be best understood by reference to the following detailed description.

According to a first aspect, the present invention is directed to a nutraceutical composition comprising dried Ziziphus mauritiana leaves, dried Lepidium sativum leaves, dried olive leaves at specific weight ratios and a specific amount of olive oil. The disclosed composition has been shown to effectively treat with minimal side effects, the common symptoms of sickle cell disease (SCD) including severe pain, bacterial infection, inflammation, wounds, fever, cough, shortness of breath.

According to a second aspect, the present invention relates to a method of treating SCD patients using the nutraceutical composition.

According to a third aspect, the present invention relates to a nutraceutical beverage based upon the disclosed composition.

Various embodiments of methods of treating SCD symptoms and methods of preparing the composition are also described in the present invention.

### DETAILED DESCRIPTION OF THE EMBODIMENTS

For the treatment of common major symptoms associated with sickle cell disease (SCD), a nutraceutical composition comprising specific amounts of dried leaves of Ziziphus mauritiana, seeds of Lepidium sativum, dried olive leaves and olive oil, may be effectively used. These major symptoms include, but are not limited to, severe pain, bacterial infection, inflammation, wounds, fever, cough, shortness of breath. In other words, the composition disclosed herein is designed to possess analgesic, anti-inflammatory, anti-bacterial, wound-healing properties and to also improve lung and breathing functions.

1. A nutraceutical composition comprising the following constituents:
(a) Ziziphus mauritiana leaves;
(b) Lepidium sativum seeds;
(c) olive leaves (Olea europaea); and
(d) olive oil;
wherein each constituent is present in an effective proportion such that, when administered to a human subject diagnosed with sickle cell disease and/or displaying symptoms thereof, said nutraceutical composition is effective in alleviating said symptoms.

2. The nutraceutical composition of claim 1, wherein (a), (b) and (c) are in dried form.

3. The nutraceutical composition of claim 1, wherein (a), (b) and (c) are in ground form.

4. The nutraceutical composition of claim 1, wherein a ratio by weight of (a) to (b) and (c) is 1:1:1.

5. The nutraceutical composition of claim 1, wherein a ratio by total weight of (a), (b) and (c) in g to total volume of (d) in ml is 1:15.

6. A method of treating symptoms of sickle cell disease, comprising administering to a human subject diagnosed with the disease and/or displaying symptoms thereof, a nutraceutical composition comprising the following constituents:
(a) Ziziphus mauritiana leaves;
(b) Lepidium sativum seeds;
(c) olive leaves (Olea europaea); and
(d) olive oil;
wherein each constituent is present in an effective proportion such that, when administered to said human subject, said nutraceutical composition is effective in alleviating said symptoms.

7. The method of claim 6, wherein (a), (b) and (c) are in dried form.

8. The method of claim 6, wherein (a), (b) and (c) are in ground form.

9. The method of claim 6, wherein a ratio by weight of (a) to (b) and (c) is x:y:z, wherein x=1 to 5, y=1 to 5 and z=1 to 5.

10. The method of claim 6, wherein a ratio by total weight of (a), (b) and (c) in g to total volume of (d) in ml is between 1:10 and 1:20.

11. The method of claim 6, wherein said nutraceutical composition is administered in a dosage of about 0.7 g per kg of body weight of the human subject, twice per day.

12. The method of claim 6, wherein said nutraceutical composition is administered for a period of at least two months.

13. The method of claim 6, wherein said administration is oral.

14. The method of claim 6, wherein said administration is topical.

15. A nutraceutical beverage comprising the following constituents:
(a) Ziziphus mauritiana leaves;
(b) Lepidium sativum seeds;
(c) olive leaves (Olea europaea);
(d) olive oil;
wherein each constituent is present in an effective proportion such that, when administered to a human subject diagnosed with sickle cell disease and/or displaying symptoms thereof, said beverage is effective in alleviating said symptoms.

16. The nutraceutical beverage of claim 15, wherein (a), (b) and (c) are in dried form.

17. The nutraceutical beverage of claim 15, wherein (a), (b) and (c) are in ground form.

18. The nutraceutical beverage of claim 15, wherein a ratio by weight of (a) to (b) and (c) is x:y:z, wherein x=1 to 5, y=1 to 5 and z=1 to 5.

19. The nutraceutical beverage of claim 15, wherein a ratio by total weight of (a), (b) and (c) in g to total volume of (d) in ml is between 1:10 and 1:20.

20. The nutraceutical beverage of claim 15, wherein said nutraceutical beverage is administered in a dosage of about 0.7 g of total weight of (a), (b), (c) and (d) per kg of body weight of the human subject, twice per day.
